(19) United States
(12) Patent Application Publication
(10) Pub. No.: US 2024/0136029 A1
(43) Pub. Date: Apr. 25, 2024

(54) THERMODYNAMIC MODELING OF UREA INCLUSION FRACTIONATION

(71) Applicant: Purdue Research Foundation, West Lafayette, IN (US)

(72) Inventors: Junli Liu, West Lafayette, IN (US); Bernard Y Tao, Lafayette, IN (US)

(21) Appl. No.: 18/328,467

(22) Filed: Jun. 1, 2023

Related U.S. Application Data
(60) Provisional application No. 63/348,035, filed on Jun. 2, 2022.

(51) Int. Cl. G16C 60/00; G06F 30/28; G16C 20/30; G16C 20/70

(52) U.S. Cl. CPC G16C 60/00; G06F 30/28; G16C 20/30; G16C 20/70

(57) ABSTRACT

A method for predicting yield and composition changes during urea inclusion fractionation to select a fatty acid methyl ester source for biofuel. The method can include heating a solid urea inclusion compound to a decomposition temperature, allowing the solid urea inclusion compound to decompose, heating again to a melting temperature, allowing the solid urea to melt, cooling to the decomposition temperature, and cooling again to a temperature below the decomposition temperature. Each heating, cooling, decomposing, and melting step can have its change in enthalpy and entropy recorded. A thermodynamic model can be calculated using the recorded changes in enthalpy and entropy. The thermodynamic model can predict a yield and composition of the solid urea inclusion compound and can be used to select a fatty acid methyl ester source using the predicted yield and composition.

### CROSS-REFERENCE TO RELATED APPLICATIONS

This application claims priority to U.S. Patent Application 63/348,035, filed Jun. 2, 2022, which is incorporated by reference herein in its entirety.

### BACKGROUND

### Field

Embodiments provided herein relate to biofuels. More particularly, embodiments provided herein relate to methods for selecting fatty acid methyl esters (FAMEs) for biofuels.

### Description of the Related Art

Elevated concerns about environmental problems from the limited available resources of fossil fuel and acting goal of net-zero emission prompted intensive research for alternative renewable energy to meet the continuously growing demand. Biodiesel can be viewed as the alternative to diesel fuels which account for approximately 24% of transportation fuel and 21% of total crude oil consumption in the United States. The annual biodiesel production has been about 1.8 billion gallons since 2018, and the primary feedstock can be soybean oil which accounts for over 60% of total feedstocks. The other feedstocks include canola oil, corn oil, poultry, and tallow. Methanol is the primary alcohol used in biodiesel production, and biodiesel consists of a mixture of fatty acid methyl esters (FAMEs) after the reactions. However, the FAMEs compositions can be constrained by the source of oils/fats. The major FAMEs shown in FIG. 1 include methyl palmitate (C16:0), methyl palmitoleate (C16:1), methyl stearate (C18:0), methyl oleate (C18:1), methyl linoleate (C18:2), and methyl linolenate (C18:3). According to the melting points, FAMEs can be separated into high melting-point FAMEs and low melting point FAMEs. In addition, FAMEs can be separated into linear saturated FAMEs and nonlinear unsaturated FAMEs according to the molecular structure. The variance in the composition of the FAMEs can significantly affect the biodiesel's qualities, such as low-temperature performance.

There is still a need, therefore, for a method of selecting FAMEs for biodiesel production that reduces low-temperature performance issues.

### SUMMARY

In at least one embodiment, a method for predicting yield and composition changes during urea inclusion fractionation to select a fatty acid methyl ester source for biofuel, comprising providing a solid urea inclusion compound having a first temperature; heating the solid urea inclusion compound to a second temperature sufficient to decompose the solid urea inclusion compound; recording a change in enthalpy and a change in entropy to provide a first recorded change in enthalpy and a first recorded change in entropy from the first temperature to the second temperature; decomposing the solid urea inclusion compound at the second temperature to produce a mixture of solid urea and liquid fatty acid methyl ester; recording a change in enthalpy and a change in entropy while decomposing the solid urea inclusion compound at the second temperature to provide a second recorded change in enthalpy and a second recorded change in entropy; heating the mixture of the solid urea and liquid fatty acid methyl ester to a third temperature sufficient to melt the urea, wherein the third temperature can be greater than the second temperature; recording a change in enthalpy and a change in entropy to provide a third recorded change in enthalpy and a third recorded change in entropy from the second temperature to the third temperature; melting the solid urea at the third temperature to produce a mixture of liquid urea and liquid fatty acid methyl ester; recording a change in enthalpy and a change in entropy while melting the solid urea to provide a fourth recorded change in enthalpy and a fourth recorded change in entropy; cooling the mixture of the liquid urea and liquid fatty acid methyl ester to the first temperature; recording a change in enthalpy and a change in entropy to provide a fifth recorded change in enthalpy and a fifth recorded change in entropy from the third temperature to the second temperature; cooling the mixture of liquid urea and liquid fatty acid methyl ester to a fourth temperature; recording a change in enthalpy and a change in entropy to provide a sixth recorded change in enthalpy and a sixth recorded change in entropy from the second temperature to the fourth temperature; calculating a thermodynamic model using the six recorded changes in enthalpies and six changes in entropies; using the thermodynamic model to predict a yield and composition of the solid urea inclusion compound; and selecting a fatty acid methyl ester source using the predicted yield and composition.

### BRIEF DESCRIPTION OF THE DRAWINGS

So that the manner in which the above recited features of the present invention can be understood in detail, a more particular description of the invention, briefly summarized above, may be had by reference to embodiments, some of which can be illustrated in the appended drawings. It can be noted, however, that the appended drawings illustrate only typical embodiments of this invention and are, therefore, not to be considered limiting of its scope, for the invention may admit to other equally effective embodiments. It can be emphasized that the figures can be not necessarily to scale and certain features and certain views of the figures can be shown exaggerated in scale or in schematic for clarity and/or conciseness.

FIG. 1 depicts illustrative structures and the melting points of major FAMEs in biodiesel.

FIG. 2 depicts a flowchart representation of a method of calculating enthalpy and entropy change.

FIG. 3 depicts a graphical representation of the relationship between molar ratios of urea to alkanes in urea inclusion compounds (“UICs”) and the number of CH₂ functional groups.

FIG. 4 depicts a graphical representation of the relationship between Δβ and the number of CH=functional groups.

FIG. 5 depicts a graphical representation of the relationship between decomposition enthalpies of UICs vs. number of CH₂ functional groups in FAMEs.

FIG. 6 depicts a graphical representation of the relationship between decomposition enthalpies of UICs vs. sum of CH₂ and CH₃ functional groups in FAMEs.

FIG. 7 depicts a graphical representation of the relationship between decomposition entropies of UICs vs. number of CH₂ functional groups in FAMEs.

FIG. 8 depicts a graphical representation of the relationship between decomposition entropies of UICs vs. sum of CH₂ and CH₃ functional groups in FAMEs.

FIG. 9 depicts a graphical representation of the relationship between decomposition enthalpies of UICs vs. number of CH═CH functional groups in FAMEs.

FIG. 10 depicts a graphical representation of the relationship between decomposition entropies of UICs vs. number of CH═CH functional groups in FAMEs.

FIG. 11 depicts a graphical representation of the relationship between decomposition temperatures of UICs formed by urea and saturated FAMEs vs. melting points of saturated FAMEs.

FIG. 12 depicts a flowchart representation of a method of cooling urea inclusion process.

FIG. 13A depicts a graphical representation of the effect of mass ratios of urea to FAME from soybean oil on cooling urea inclusion fractionation as measured by yield.

FIG. 13B depicts a graphical representation of the effect of mass ratios of urea to FAME from soybean oil on cooling urea inclusion fractionation as measured by C16:0.

FIG. 13C depicts a graphical representation of the effect of mass ratios of urea to FAME from soybean oil on cooling urea inclusion fractionation as measured by C18:0.

FIG. 13D depicts a graphical representation of the effect of mass ratios of urea to FAME from soybean oil on cooling urea inclusion fractionation as measured by C18:1.

FIG. 13E depicts a graphical representation of the effect of mass ratios of urea to FAME from soybean oil on cooling urea inclusion fractionation as measured by C18:2.

FIG. 13F depicts a graphical representation of the effect of mass ratios of urea to FAME from soybean oil on cooling urea inclusion fractionation as measured by C18:3.

FIG. 14A depicts a graphical representation of the effect of mass ratios of methanol to FAME from soybean oil on cooling urea inclusion fractionation as measured by yield.

FIG. 14B depicts a graphical representation of the effect of mass ratios of methanol to FAME from soybean oil on cooling urea inclusion fractionation as measured by C16:0.

FIG. 14C depicts a graphical representation of the effect of mass ratios of methanol to FAME from soybean oil on cooling urea inclusion fractionation as measured by C18:0.

FIG. 14D depicts a graphical representation of the effect of mass ratios of methanol to FAME from soybean oil on cooling urea inclusion fractionation as measured by C18:1.

FIG. 14E depicts a graphical representation of the effect of mass ratios of methanol to FAME from soybean oil on cooling urea inclusion fractionation as measured by C18:2.

FIG. 14F depicts a graphical representation of the effect of mass ratios of methanol to FAME from soybean oil on cooling urea inclusion fractionation as measured by C18:3.

FIG. 15A depicts a graphical representation of the effect of carbon chain length of solvent on cooling urea inclusion fractionation as measured by yield.

FIG. 15B depicts a graphical representation of the effect of carbon chain length of solvent on cooling urea inclusion fractionation as measured by C16:0.

FIG. 15C depicts a graphical representation of the effect of carbon chain length of solvent on cooling urea inclusion fractionation as measured by C18:0.

FIG. 15D depicts a graphical representation of the effect of carbon chain length of solvent on cooling urea inclusion fractionation as measured by C18:1.

FIG. 15E depicts a graphical representation of the effect of carbon chain length of solvent on cooling urea inclusion fractionation as measured by C18:2.

FIG. 15F depicts a graphical representation of the effect of carbon chain length of solvent on cooling urea inclusion fractionation as measured by C18:3.

FIG. 16A depicts a graphical representation of the effect of the composition of FAME on cooling urea inclusion fractionation yields.

FIG. 16B depicts a graphical representation of the effect of the composition of FAME on cooling urea inclusion fractionation FAME from soybean oil.

FIG. 16C depicts a graphical representation of the effect of the composition of FAME on cooling urea inclusion fractionation FAME from safflower oil.

FIG. 16D depicts a graphical representation of the effect of the composition of FAME on cooling urea inclusion fractionation FAME from grapeseed oil.

FIG. 16E depicts a graphical representation of the effect of the composition of FAME on cooling urea inclusion fractionation FAME from flaxseed oil.

FIG. 16F depicts a graphical representation of the effect of the composition of FAME on cooling urea inclusion fractionation FAME from palm oil.

FIG. 16G depicts a graphical representation of the effect of the composition of FAME on cooling urea inclusion fractionation FAME from waste cooking oil.

FIG. 16H depicts a graphical representation of the effect of the composition of FAME on cooling urea inclusion fractionation FAME from chicken fat.

### DETAILED DESCRIPTION

It can be to be understood that the following disclosure describes several exemplary embodiments for implementing different features, structures, or functions of the invention. Exemplary embodiments of components, arrangements, and configurations can be described below to simplify the present disclosure; however, these exemplary embodiments can be provided merely as examples and can be not intended to limit the scope of the invention. Additionally, the present disclosure can repeat reference numerals and/or letters in the various embodiments and across the figures provided herein. This repetition can be for the purpose of simplicity and clarity and does not in itself dictate a relationship between the various embodiments and/or configurations. Moreover, the exemplary embodiments presented below can be combined in any combination of ways, i.e., any element from one exemplary embodiment can be used in any other exemplary embodiment, without departing from the scope of the disclosure.

Additionally, certain terms can be used throughout the following description and claims to refer to particular components. As one skilled in the art will appreciate, various entities can refer to the same component by different names, and as such, the naming convention for the elements described herein can be not intended to limit the scope of the invention, unless otherwise specifically defined herein. Further, the naming convention used herein can be not intended to distinguish between components that differ in name but not function.

Furthermore, in the following discussion and in the claims, the terms “including” and “comprising” can be used in an open-ended fashion, and thus should be interpreted to mean “including, but not limited to.”

The term “or” can be intended to encompass both exclusive and inclusive cases, i.e., “A or B” can be intended to be synonymous with “at least one of A and B,” unless otherwise expressly specified herein.

The indefinite articles “a” and “an” refer to both singular forms (i.e., “one”) and plural referents (i.e., one or more) unless the context clearly dictates otherwise. For example, embodiments using “an olefin” include embodiments where one, two, or more olefins can be used, unless specified to the contrary or the context clearly indicates that only one olefin can be used.

Unless otherwise indicated herein, all numerical values can be “about” or “approximately” the indicated value, meaning the values take into account experimental error, machine tolerances and other variations that would be expected by a person having ordinary skill in the art. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for making the measurement.

Each of the appended claims defines a separate invention, which for infringement purposes can be recognized as including equivalents to the various elements or limitations specified in the claims. Depending on the context, all references to the “invention” may in some cases refer to certain specific embodiments only. In other cases, it will be recognized that references to the “invention” will refer to subject matter recited in one or more, but not necessarily all, of the claims. Each of the inventions will now be described in greater detail below, including specific embodiments, versions, and examples, but the inventions can be not limited to these embodiments, versions, or examples, which can be included to enable a person having ordinary skill in the art to make and use the inventions when the information in this disclosure can be combined with publicly available information and technology.

As used herein, “wt %” means weight percent, “mol %” means mole percent, “vol %” means volume percent, and all molecular weights, e.g., Mw, Mn, Mz, can be in units of g/mol, unless otherwise noted. Furthermore, all molecular weights can be Mw unless otherwise noted.

As used herein, “consisting essentially of” means that the described/claimed composition does not include any other components that will materially alter its properties by any more than 5% of that property, and in any case, does not include any other component to a level greater than 3 wt %.

As used herein, a “fatty acid methyl ester source” means any material that contains sufficient FAMEs that can be processed by any means capable of producing biofuel, biofuel components, biofuel derivatives, and/or the like. A fatty acid methyl ester source can include soybean oil, safflower oil, grape seed oil, flaxseed oil, cooking oil, chicken fat, palm oil, and/or the like, or any combination thereof.

Though biodiesel owns renewability and sustainability, its poor low-temperature performance constrained the utilization scopes. With the surrounding temperature decreasing, FAMEs with high-melting points tend to form solid crystals, and these crystals can block filters and lines. Four standards in ASTM D 6751 can be used to evaluate the low-temperature performance: cloud point (CP), pour point (PP), cold filter plugging point (CFPP), and low-temperature flow test (LTFT). CP appropriately described the physical phase change concerning the temperature compared to other standards. CP of biodiesel ranged from −5 to 21° C. for various feedstocks. Therefore, biodiesel generally had a higher CP than #2 diesel−8 to −27° C.) and #1 diesel (−40° C.). Low-temperature performance needs to be improved for enlargening biodiesel utilization.

As the saturated FAMEs significantly affect the low-temperature performance, the effective way to improve the low-temperature performance can be to remove the saturated FAMEs. The current methods to remove the saturated FAMEs can be thermal crystallization, solvent extraction, distillation, and urea inclusion fractionation. Thermal crystallization can be performed to remove the high melting points components with the decrease in the surrounding temperature. Also, it can be an energy-intensive process with extending operation time to reach the equilibrium for the separation. Solvent extraction combined with thermal crystallization can be applied to reduce CP with different solvents, such as hexane and isopropanol. The disadvantages of this method can be the high amount of consumption of solvents, difficulty process control from the surrounding temperature change, and CP reduction limitations even with the decreasing bath temperatures. Another phase-changing method can be biodiesel distillation fractionation. In addition to high energy consumption, this operation can be inhibited by the poor separation of the C18 FAMEs due to the difference in boiling points for these FAMEs can be not significant. For all the phase change methods, saturated FAMEs can be a residue in low CP portion due to phase equilibrium. As the saturated FAMEs with high melting points, these methods can have limited CP reduction components.

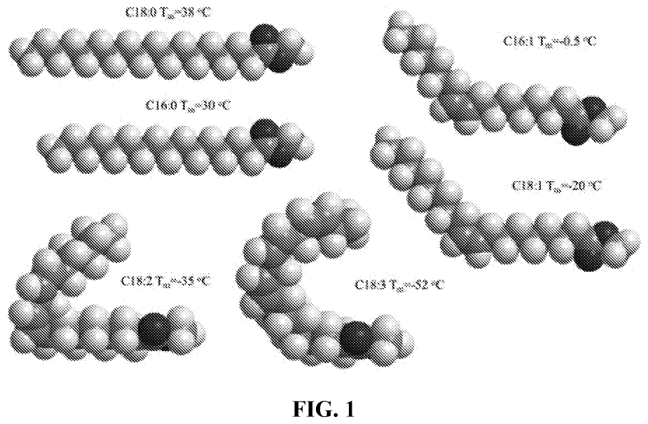

Compared to these fractionation methods, urea inclusion can be more efficient in removing the saturated FAMEs. FAMEs and fatty acids can have similar structures with different functional groups: ester vs. carboxylic acid. Since C16:0 and C18:0 can be linear molecules, they preferentially form urea inclusion compounds (UICs) vs. nonlinear C16:1, C18:1, C18:2, and C18:3 molecules (see FIG. 1). Therefore, urea inclusion fractionation can separate saturated/unsaturated FAMEs, and the FAMEs in liquid solution (L-FAMEs) become unsaturated enriched. The unsaturated enriched FAMEs result in significantly reduced CPs, suitable for cold weather fuels. Urea inclusion can be applied to fractionate the biodiesel from various oils/fats reduce the CP. Here, a thermodynamic model can be proposed according to phase equilibrium and non-ideal behavior predicted by the modified UNIFAC model to predict the urea inclusion process robustly.

Traditional heterogeneous phase equilibrium shows the transport of the components from one phase to another. There can be no compound formation in the phase change for the traditional heterogeneous phase equilibrium. In urea inclusion fractionation, urea forms solid compounds with guest molecules (as shown in Eq.1). At equilibrium, urea and guest molecules in the liquid phase can be equilibrated with the solid compounds. Therefore, the formation of urea inclusion compounds can be a unique phase equilibrium. In this section, the principles of traditional phase equilibrium can be applied to model urea inclusion fractionation.

βU+Fᵢ=U_{β}Fᵢ Eq.1

Where U can be Urea; Fᵢ can be the i^{th} type of FAME; β can be the mole ratio of urea to FAME in UIC.

According to the principles of heterogeneous phase equilibrium, there can be three relationships at equilibrium. The temperature in solid phase can be equal to the temperature in liquid phase:

T_{S}=T_{L} Eq. 2

The pressure in solid phase can be equal to the pressure in liquid phase:

P_{S}=P_{L} Eq. 3

The chemical potential of UIC formed by urea with i^{th} type of FAME in solid phase can be equal to that in liquid phase:

μ_{UIC,i}^{S}=μ_{UIC,i}^{L} Eq. 4

The chemical potentials of UIC formed by urea with i^{th} type of FAME in solid phase and liquid phase can be shown in Eq. 5 and Eq. 6.

μ_{UIC,i}^{S}(T,P)=μ_{UIC,i}^{S,o}(T,P)+RT ln(α_{UIC,i}^{S} Eq. 5

μ_{UIC,i}^{L}(T,P)=μ_{UIC,i}^{L,o}(T,P)+RT ln(α_{UIC,i}^{L} Eq. 6

μ_{UIC,i}^{S}(T,P): Chemical potential of UIC formed by urea with i^{th} type of FAME in solid phase at temperature T and pressure P
μ_{UIC,i}^{L}(T,P): Chemical potential of UIC formed by urea with i^{th} type of FAME in liquid phase at temperature T and pressure P
μ_{UIC,i}^{S,o}(T,P): Standard chemical potential of UIC formed by urea with i^{th} type of FAME in solid phase at temperature T and pressure P
μ_{UIC,i}^{L,o}(T,P): Standard chemical potential UIC formed by urea with i^{th} type of FAME in liquid phase at temperature T and pressure P
α_{UIC,i}^{S}(T,P): Activity of UIC formed by urea with i^{th} type of FAME in solid phase
α_{UIC,i}^{L,o}(T,P): Activity of UIC formed by urea with i^{th} type of FAME in liquid phase

There can be no urea inclusion compound in liquid phase. Urea inclusion compound decomposes to urea and FAMEs in liquid phase. Therefore, the chemical potential of urea inclusion compounds in liquid phase can be shown in Eq.7.

μ_{UIC,i}^{L}(T,P)=μ_{U}^{L,o}(T,P)+βμᵢ^{L,o}+βRT ln(α_{U}^{L})+RT ln(αᵢ^{L}) Eq. 7

μ_{U}^{L,o}(T,P): Standard chemical potential of urea in liquid phase at temperature T and pressure P
μᵢ^{L,o}(T,P): Standard chemical potential of i^{th} type of FAMEs in liquid phase at temperature T and pressure P
α_{U}^{L}: Activity of urea in liquid phase
αᵢ^{L}: Activity of i^{th} type of FAMEs in liquid phase

Eq.5 and 7 can be substituted into Eq.4, then:

μ_{UIC,i}^{S,o}(T,P)+RT ln(α_{UIC,i}^{S})=βμ_{U}^{L,o}(T,P)+μᵢ^{L,o}(T,P)+βRT ln(α_{U}^{L})+RT ln(αᵢ^{L}) Eq. 8

The activity of urea inclusion compound formed by urea with i^{th} type of FAMEs in solid phase and the activities of urea and i^{th} type of FAMEs in liquid phase can have the following relationship as shown in Eq. 9 and Eq. 10.

−RT ln((α_{U}^{L})^{β}αᵢ^{L}/α_{UIC,i}^{S})=Δμ_{UIC,i}^{o}(T,P) Eq. 9

μ_{UIC,i}^{o}(T,P)=βμ_{U}^{L,o}(T,P)+μᵢ^{L,o}(T,P)−μ_{UIC,i}^{S,o}(T,P) Eq. 10

The chemical potential and the change of chemical potential cannot be directly calculated. To use the thermodynamic model to describe the formation of urea inclusion compounds, the chemical potential should be determined. The change of chemical potential from liquid phase to solid phase can be calculated by Eq. 11.

Δμ_{UIC,i}^{o}(T,P)=ΔH_{m,UIC,i}^{o}(T,P)−TΔS_{m,UIC,i}^{o}(T,P) Eq. 11

ΔH_{m,UIC,i}^{o}(T,P): Molar enthalpy change of UIC formed by urea with i^{th} type of FAME from liquid to solid
ΔS_{m,UIC,i}^{o}(T,P): Molar entropy change of UIC formed by urea with i^{th} type of FAME from liquid to solid

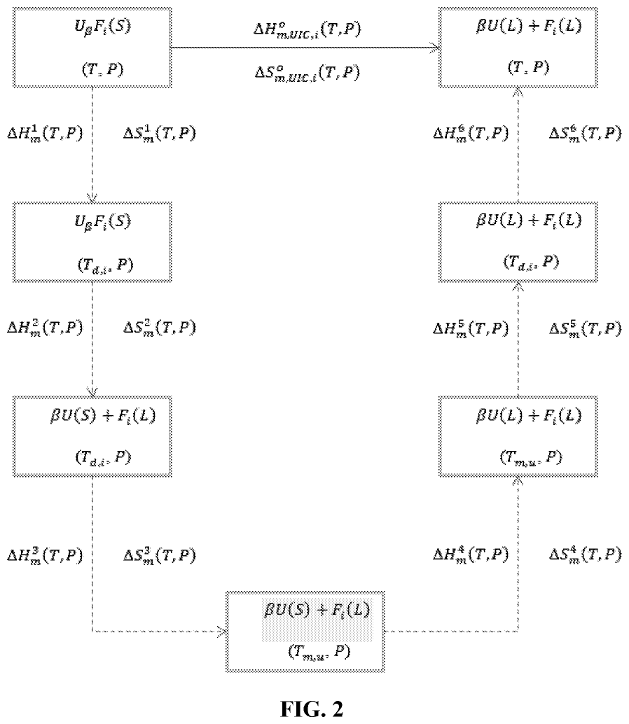

It can be impossible to obtain the enthalpy and entropy changes directly. Since these can be state variables, the enthalpy change and entropy change can be only related to the initial and final state of the component. Therefore, a route can be designed to calculate the enthalpy and entropy changes (as shown in FIG. 2). The designed thermodynamic route to calculate the enthalpy and entropy change consists of 6 steps. These steps can be under constant pressure. The solid urea inclusion compound formed by urea and fatty acid methyl ester can be heated from temperature T to its decomposition (T_{d,i}) temperature in the first step and the enthalpy change (ΔHₘ¹(T,P)) and entropy change (ΔSₘ¹(T,P)) of this step can be shown in Eq. 12 and 13, respectively. At the decomposition temperature, the solid urea inclusion compound decomposes to solid urea and liquid fatty acid methyl ester in the second step. The enthalpy change (ΔHₘ²(T,P)) and entropy change (ΔSₘ²(T,P)) of this step can be shown in Eq. 14 and 15, respectively. In the third step, the mixture of solid urea and liquid fatty acid methyl ester can be heated from the melting point of urea (T_{m,u}). The enthalpy change (ΔHₘ³(T,P)) and entropy change (ΔSₘ³(T,P)) of the third step can be shown in Eq. 16 and 17, respectively. The mixture of solid urea and liquid fatty acid methyl ester becomes liquid urea and liquid fatty acid methyl ester at the melting point of urea. The enthalpy change (ΔHₘ⁴(T,P)) and entropy change (ΔSₘ⁴(T,P)) of the fourth step can be shown in Eq. 18 and 19, respectively. In the fifth step, the liquid urea and liquid FAMEs mixture can be chilled to the decomposition temperature. The enthalpy change (ΔHₘ⁵(T,P)) and entropy change (ΔSₘ⁵(T,P)) of the fifth step can be shown in Eq. 20 and 21, respectively. In the sixth step, the mixture of liquid urea and liquid fatty acid methyl ester can be chilled to the temperature (T). The enthalpy change (ΔHₘ⁶(T,P)) and entropy change (ΔSₘ⁶(T,P)) of the fifth step can be shown in Eq. 22 and 23, respectively.

ΔHₘ¹(T,P)=∫_{T}^{T}^{_{d,i}}C_{p,m,UIC,i}^{S}dT eq. 12

ΔSₘ¹(T,P)=∫_{T}^{T}^{_{d,i}}(C_{p,m,UIC,i}^{S}/T)dT Eq. 13

ΔHₘ²(T,P)=Δ_{dec}H_{m,i} Eq. 14

ΔSₘ²(T,P)=Δ_{dec}H_{m,i}/T_{d,i} Eq. 15

ΔHₘ³(T,P)=∫_{T}_{_{d,i}}^{T}^{_{m,u}}((C_{p,m,i}^{L}+βC_{p,m,u}^{S})dT Eq. 16

ΔSₘ³(T,P)=∫_{T}_{_{d,i}}^{T}^{_{m,u}}((C_{p,m,i}^{L}+βC_{p,m,u}^{S})/T)dT Eq. 17

ΔHₘ⁴(T,P)=βΔ_{fus}H_{m,u} Eq. 18

ΔSₘ⁴(T,P)=βΔ_{fus}H_{m,u}/T_{m,u} 19

ΔH_{m,i}⁵(T,P)=−∫_{T}_{_{d,i}}^{T}^{_{m,u}}(C_{p,m,i}^{L}+βC_{p,m,u}^{L})dT Eq. 20

ΔH_{m,i}⁵(T,P)=−∫_{T}_{_{d,i}}^{T}^{_{m,u}}((C_{p,m,i}^{L}+βC_{p,m,u}^{L})/T)dT Eq. 21

ΔHₘ⁶(T,P)=−∫_{T}^{T}^{_{m,u}}(C_{p,m,i}^{L}+βC_{p,m,u}^{L})dT Eq. 22

ΔHₘ⁶(T,P)=−∫_{T}^{T}^{_{m,u}}((C_{p,m,i}^{L}+βC_{p,m,u}^{L})/T)dT Eq. 23

C_{p,m,UIC,i}^{S}: Molar heat capacity of UIC formed by urea and i^{th} type of FAME at constant pressure
C_{p,m,u}^{S}: Molar heat capacity of solid urea at constant pressure
C_{p,m,u}^{L}: Molar heat capacity of liquid urea at constant pressure
C_{p,m,i}^{L}: Molar heat capacity of liquid i^{th} type of FAMEs at constant pressure
Δ_{dec}H_{m,i}: Molar decomposition enthalpy of UIC formed by urea and it type of FAME
Δ_{fus}H_{m,u}: Molar fusion enthalpy of urea

According to thermodynamic state variables, there exist the following two relations as shown in Eq. 24 and 25.

ΔH_{m,i}^{o}(T,P)=ΔHₘ¹(T,P)+ΔHₘ²(T,P)+ΔHₘ³(T,P)+ΔHₘ⁴(T,P)+ΔHₘ⁵(T,P)+ΔHₘ⁶(T,P) Eq. 24

ΔS_{m,i}^{o}(T,P)=ΔSₘ¹(T,P)+ΔSₘ²(T,P)+ΔSₘ³(T,P)+ΔSₘ⁴(T,P)+ΔSₘ⁵(T,P)+ΔSₘ⁶(T,P) Eq.25

According to Eq. 12 through 25, the enthalpy change and entropy change of component i from liquid to solid can be shown in Eq. 26 and 27.

ΔH_{m,i}^{o}(T,P)=∫_{T}^{T}^{_{d,i}}ΔC_{p,m,i}dT+∫_{T}_{_{d,i}}^{T}^{_{m,u}}(βΔC_{p,m,u})dT+βΔ_{fus}H_{m,u}+Δ_{dec}H_{m,i} Eq. 26

ΔS_{m,i}^{o}(T,P)=∫_{T}^{T}^{_{d,i}}(ΔC_{p,m,i}/T)dT+∫_{T}_{_{d,i}}^{T}^{_{m,u}}(βΔC_{p,m,u}/T)dT+βΔ_{fus}H_{m,u}/T_{m,u}+Δ_{dec}H_{m,i}/T_{d,i} Eq. 27

ΔC_{p,m,u}: Molar heat capacity difference of urea at constant pressure in solid and liquid

ΔC_{p,m,u}=C_{p,m,u}^{S}−C_{p,m,u}^{L} Eq. 28

ΔC_{p,m,i}: Molar heat capacity difference at constant pressure between solid UIC and the mixture of i^{th} type of liquid FAME and solid urea

ΔC_{p,m,i}=C_{p,m,UIC,i}^{S}−C_{p,m,i}^{L}−βC_{p,m,u}^{L} Eq. 29

According to Eq. 9, 11, 26 and 27, the thermodynamic model to model the formation of urea inclusion compound can be shown in Eq. 30.

R ln((α_{U}^{L})^{β}αᵢ^{L}/α_{UIC,i}^{S})=∫_{T}^{T}^{_{d,i}}(ΔC_{p,m,i}/T)dT−(∫_{T}^{T}^{_{d,i}}ΔC_{p,m,i}dT)/T+∫_{T}_{_{d,i}}^{T}^{_{m,u}}(βΔC_{p,m,u}/T)dT−(∫_{T}_{_{d,i}}^{T}^{_{m,u}}(βΔC_{p,m,u})dT)/T+βΔ_{fus}H_{m,u}(1/T_{m,u}−1/T)+Δ_{dec}H_{m,i}(1/T_{d,i}−1/T) Eq. 30

According to the definition of activity, the thermodynamic model becomes:

R ln((γ_{U}^{L}x_{U}^{L})^{β}γᵢ^{L}xᵢ^{L}/(γ_{UIC,i}^{S}x_{UIC,i}^{S}))=∫_{T}^{T}^{_{d,i}}(ΔC_{p,m,i}/T)dT−(∫_{T}^{T}^{_{d,i}}ΔC_{p,m,i}dT)/T+∫_{T}_{_{d,i}}^{T}^{_{m,u}}(βΔC_{p,m,u}/T)dT−(∫_{T}_{_{d,i}}^{T}^{_{m,u}}(βΔC_{p,m,u})dT)/T+βΔ_{fus}H_{m,u}(1/T_{m,u}−1/T)+Δ_{dec}H_{m,i}(1/T_{d,i}−1/T) Eq. 31

If the capacity differences can be negligible, the thermodynamic model becomes:

R ln((γ_{U}^{L}x_{U}^{L})^{β}γᵢ^{L}xᵢ^{L}/(β_{UIC,i}^{S}x_{UIC,i}^{S}))=βΔ_{fus}H_{m,u}(1/T_{m,u}−1/T)+Δ_{dec}H_{m,i}(1/T_{d,i}−1/T) Eq. 32

The activity coefficient of the component in the mixture of FAMEs can be calculated according to the modified UNIFAC model. For one type of urea inclusion compound, Eq. 33 can be used to calculate the solubility of urea inclusion compound in the solvent.

R ln(γᵢ^{L}xᵢ^{L}(γ_{U}^{L}x_{U}^{L})β)=∫_{T}^{T}^{_{d,i}}(ΔC_{p,m,i}/T)dT−(∫_{T}^{T}^{_{d,i}}ΔC_{p,m,i}dT)/T+∫_{T}_{_{d,i}}^{T}^{_{m,u}}(βΔC_{p,m,u}/T)dT−(∫_{T}_{_{d,i}}^{T}^{_{m,u}}(βΔC_{p,m,u})dT)/T+β_{fus}H_{m,u}(1/T_{m,u}−1/T)+Δ_{dec}H_{m,i}(1/T_{d,i}−1/T) Eq. 33

For multiply types of UICs, Eq. 31 can be used to calculate the composition of liquid phase and solid phase at the equilibrium state.

The activity coefficients in the model can be calculated according to the modified UNIFAC model which can be expressed in Eq. 34.

ln γᵢ=ln γᵢ^{GS}+ln γᵢ^{GI} Eq. 34

ln γᵢ^{GS}=1−Vᵢ′+ln Vᵢ′−5_{q}_{ᵢ}(1−Vᵢ/Fᵢ+ln(Vᵢ/Fᵢ)) Eq. 35

Vᵢ′=rᵢ^{3/4}/Σⱼxⱼrⱼ^{3/4} Eq. 36

Vᵢ=rᵢΣⱼxⱼrⱼ Eq.37

rᵢ=Σᵢvₖᵢδᵢ Eq. 38

Fᵢ=qᵢ/Σⱼxⱼqⱼ Eq. 39

qᵢ=ΣᵢδₖᵢQᵢ Eq. 40

xⱼ: Molar Fraction of component j
δₖ: Volume parameter of group k
Qₖ: Surface area parameter of group k
vₖᵢ: Number of group k in component i

The effect of the group interaction on the activity coefficient can be shown in Eq. 41.

ln γᵢ^{GI}=Σₖδ_{k,i}(ln ηₖ−ln η_{K}ⁱ) Eq. 41

ln ηₖ can be the group k contribution on the activity coefficient through the group interaction (as shown in Eq. 42) and ln ηₖⁱ can be the group k contribution on the activity coefficient through the group interaction in the pure component i (as shown in Eq. 43).

ln ηₖ=5Qₖ(1−ln(Σₘθₘτₘₖ)−Σₗ(θₗτₖₗ)/Σⱼθⱼτⱼₗ) Eq. 42

ln ηₖⁱ=5Qₖ(1−ln(Σₘθₘτₘₖ)−Σₗ(θₗτₖₗ)/Σⱼθⱼτⱼₗ)(for xᵢ=1) Eq. 43

θₘ=QₘXₘ/ΣₙQₙXₙ Eq. 44

Xₘ=Σⱼvₘⱼxⱼ/ΣₙΣⱼvₘⱼxⱼ Eq. 45

τₘ=exp(−Aⱼᵢ/T−Bⱼᵢ−CⱼᵢT) Eq. 46

Aⱼᵢ: Group interaction parameter
Bⱼᵢ: Group interaction parameter
Cⱼᵢ: Group interaction parameter

The molar ratios of urea FAMEs can increase with the increase in the carbon chain length of the saturated FAMEs. With the same carbon chain length of FAMEs, the molar ratios of urea to FAMEs can decrease with the increase in cis carbon double bonds. The molar ratios of urea to FAMEs in UICs can be shown in Table 2.

TABLE 2
Molar Ratio of Urea to FAMEs in Urea Inclusion Compounds

| | C12:0 | C14:0 | C16:0 | C18:0 | C18:1 | C18:2 |
|---|---|---|---|---|---|---|
| Molar Ratio | 10.9 | 12.2 | 13.5 | 14.8 | 14.5 | 14.2 |

Some molar ratios of urea to FAMEs can be determined in this disclosure (Table. 2) or can be known from the literature. However, some molar ratios of urea to FAMEs in UICs can be still unknown. In this section, the functional group contribution on the ratios can be studied and the values of the group contribution on the ratios can be used to calculate the ratios of urea to FAMEs in UICs.

For UICs formed by urea and FAMEs, the functional groups in FAMEs include CH₃, CH₂, COO and CH═. The molar ratios of urea to FAMEs in UICs can be calculated according to Eq. 47.

β=ΣNᵢEᵢ Eq. 47

Where Nᵢ and Eᵢ can be the number of group i in the FAMEs and the effect of group i on the molar ratio of urea to FAMEs in UICs, respectively.

According to the alkane structures, there can be two CH₃ groups in alkanes, but the number of CH₂ varies with the carbon chain length of the alkane. According to the group contribution model, the molar ratio of urea to alkane can be shown in Eq. 48.

β=E_{CH}_{₂}N_{CH}_{₂}+E_{CH}_{₃}N_{CH}_{₃}Eq. 48

E_{CH}_{₂}: Effect of CH₂ on the molar ratio
N_{CH}_{₂}: Number of CH₂ in the molecule
E_{CH}_{₃}: Effect of CH₃ on the molar ratio
N_{CH}_{₃}: Number of CH₃ in the molecule

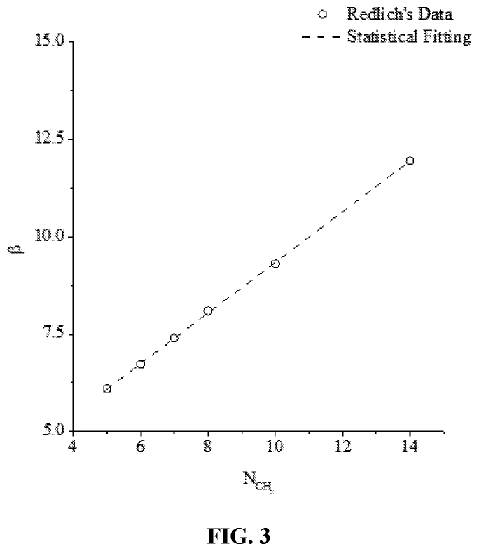

According to the plot of molar ratio with the number of CH₂ (as shown in FIG. 3), there can be a linear relationship between the molar ratio and the number of CH₂ as seen in Eq. 49.

β=0.649N_{CH}_{₂}+2.855 Eq. 49

In Eq 49, the slope of the linear relationship shows the effect of CH₂ group on the molar ratios of urea to FAMEs and the molar ratios of urea to FAMEs increase at 0.649 per CH₂ group. And, the intercept of the linear relationship indicates the effect of CH₃ group on the molar ratios of urea to FAMEs. Since there can be two CH₃ groups in alkanes, the molar ratios of urea to FAMEs increase at 1.4275 per CH₃ group.

The number of functional groups in FAMEs in this disclosure can be listed in Table 3. Compared to the saturated FAMEs to the alkanes, their difference can be the esters functional group. Moreover, the molar ratio of urea to saturated FAMEs can be calculated from Eq. 50 according to the group contribution model.

β=E_{CH}_{₂}N_{CH}_{₂}+E_{CH}_{₃}N_{CH}_{₃}+E_{coo}N_{coo} Eq. 50

E_{coo}: Effect of COO on the molar ratio
N_{coo}: Number of COO in the molecule

There can be only one ester functional group in the saturated FAMEs, and Eq. 50 becomes:

β=E_{CH}_{₂}N_{CH}_{₂}+E_{CH}_{₃}N_{CH}_{₃}+E_{coo} Eq. 51

The effect of the ester functional group on the molar ratio can be the difference between the measured molar ratio and the calculated molar ratio from the CH₂ and CH₃ contribution. One ester functional group makes the molar ratios rise 1.558.

TABLE 3
Number of Functional Groups in FAMEs

| | C12:0 | C14:0 | C16:0 | C16:1 | C18:0 | C18:1 | C18:2 | C18:3 |
|---|---|---|---|---|---|---|---|---|
| CH₃ | 2 | 2 | 2 | 2 | 2 | 2 | 2 | 2 |
| CH₂ | 10 | 12 | 14 | 12 | 16 | 14 | 12 | 10 |
| CH═ | 0 | 0 | 0 | 2 | 0 | 2 | 4 | 6 |
| COO | 1 | 1 | 1 | 1 | 1 | 1 | 1 | 1 |

The molar ratio of the unsaturated FAMEs can be calculated from Eq.52 according to the group contribution model.

β=E_{CH}_{₂}N_{CH}_{₂}+E_{CH}_{₃}N_{CH}_{₃}+E_{coo}N_{coo}+E_{CH═}N_{CH═} Eq. 52

E_{CH═}: Effect of CH═ on the molar ratio
N_{CH═}: Number of CH═ in the molecule

The difference between the measured molar ratio and the molar ratio from CH₂, CH₃ and COO contribution can be defined in Eq. 53.

Δβ=β−(E_{CH}_{₂}N_{CH}_{₂}+E_{CH}_{₃}N_{CH}_{₃}+E_{coo}N_{coo}) Eq. 53

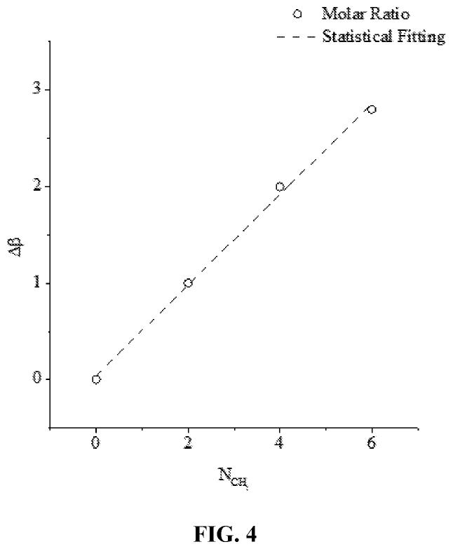

As seen in FIG. 4, as can be linearly related to the number of CH═ and the relationship can be shown in Eq. 54.

Δβ=0.469_{CH}=+0.043 Eq. 54

Where C_{CH}═ can be the number of CH=functional groups in the guest molecules.

The effects of the functional groups on the molar ratio of urea to FAMEs follow the order: COO>CH₃>CH₂>CH═. The order indicates the length of the functional groups in the direction of the urea host structure axis. The group contribution values to predict the molar ratio of urea to FAMEs can be shown in Table 4.

TABLE 4
Group Contribution Values on the Molar Ratio of Urea to FAME

| | CH₃— | —CH₂— | —CH═ | —COO— |
|---|---|---|---|---|
| Molar Ratio | 1.4275 | 0.649 | 0.469 | 1.558 |

The decomposition temperatures and enthalpies of urea inclusion compounds should be determined so that the proposed model can be used to predict the urea inclusion fractionation. The decomposition temperature and enthalpy exist a relationship (as shown in Eq. 55).

Δ_{Dec}Hₘ=Δ_{Dec}SₘT_{D} Eq. 55

Δ_{Dec}Hₘ: Molar decomposition enthalpy of urea inclusion compound
Δ_{Dec}Sₘ: Molar decomposition entropy of urea inclusion compound
T_{D}: Decomposition temperature of urea inclusion compound

A group contribution method can be applied to predict the decomposition entropy (as shown in Eq. 56).

Δ_{Dec}Sₘ=ΣCᵢωᵢ Eq. 56

ωᵢ: Group contribution value of group i on the decomposition entropy
Cᵢ: Number of Group i in the molecule

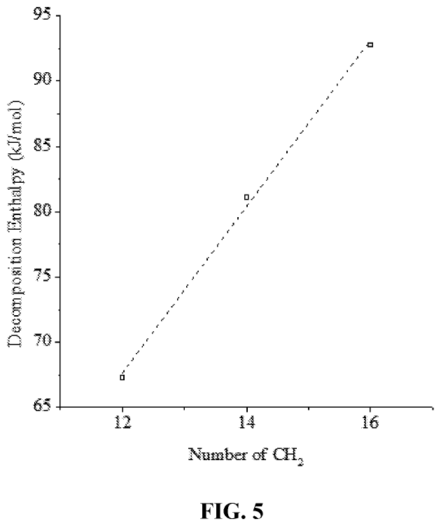

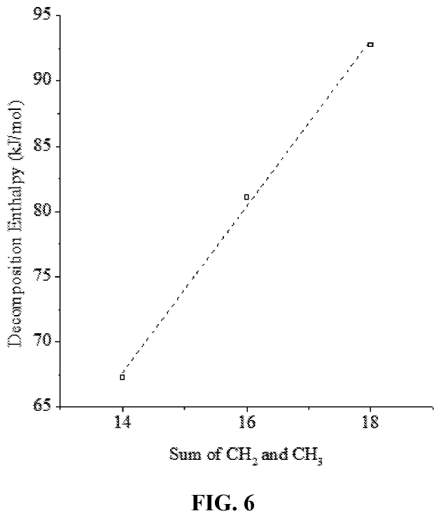

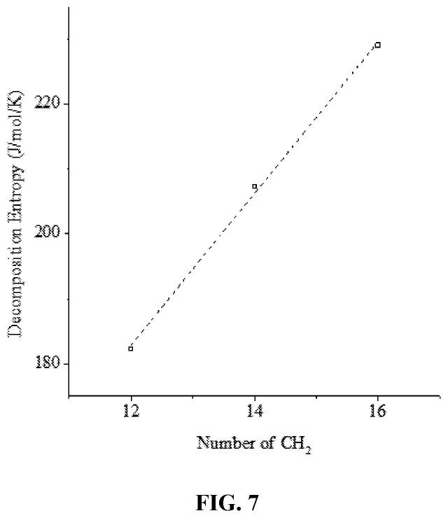

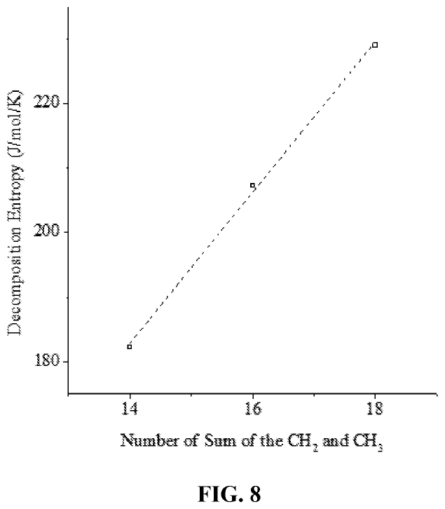

The functional groups in FAMEs can be CH₃, CH₂, CH═ and COO. The number of the functional groups in the FAMEs can be shown in Table 3. According to the published decomposition enthalpy of urea inclusion compounds formed by urea and saturated FAMEs, the decomposition enthalpies linearly increase with the increase in CH₂ in FAMEs (as shown in FIG. 5 and Eq. 57). The decomposition enthalpies of UICs formed by urea and saturated FAMEs linearly increased with the increase in the sum of CH₂ and CH₃ (as shown in FIG. 6 and Eq. 58). According to Eq. 57 and 58, CH₂ has the same contribution on the decomposition enthalpies as the CH₃ and the group contribution value can be 6374.5 J/mol. By the way, the group contribution of ester functional group (COO) on the decomposition enthalpy can be −21596.7 J/mol. Based on the reported data of the decomposition temperatures and decomposition enthalpies of UICs formed by urea and saturated FAMEs, the decomposition entropies can be calculated and linearly increases with the number of CH₂ group (as shown in FIG. 7 and Eq. 59). Also, the decomposition entropies of UICs linearly increase with the sum of CH₂ and CH₃ (as shown in FIG. 8 and Eq. 60). According to Eq. 59 and 60, the CH₂ functional group has the same contribution on the decomposition entropy as CH₃ and the group contribution value can be 11.684 J/mol/K. Also, the group contribution of ester functional group (COO) on the decomposition entropy can be 19.278 J/mol/K.

Δ_{Dec}Hₘ=6374.5C_{CH}_{₂}−8847.7 Eq. 57

Δ_{Dec}Hₘ=6374.5(C_{CH}_{₂}+C_{CH}_{₃})−21596.7 Eq. 58

Δ_{Dec}Sₘ=11.684C_{CH}_{₂}+42.646 Eq. 59

Δ_{Dec}Sₘ=11.684(C_{CH}_{₂}+C_{CH}_{₃})+19.278 Eq. 60

Where C_{CH}_{₂} can be the number of CH₂ functional group in FAMEs.

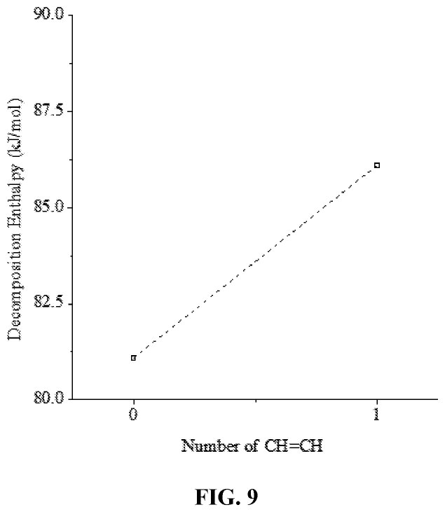

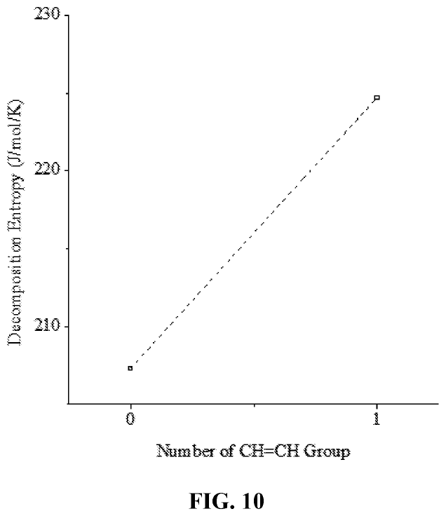

As seen in Table 3, methyl palmitate and methyl oleate can have the same number of functional groups, except the CH═CH. Because of the limited available data on the decomposition enthalpies of UICs formed by urea and the unsaturated FAMEs, the group contribution of CH═CH on the decomposition enthalpies can be calculated from the difference in the decomposition enthalpy of UICs formed by urea and methyl oleate and that of UICs formed by urea and methyl palmitate (as shown in FIG. 9 and Eq. 61). Also, the contribution of CH═CH to the decomposition entropies can be the difference in the decomposition entropy of UICs formed by urea and oleate and that UICs formed by urea and methyl palmitate (as shown in FIG. 10 and Eq. 62). The group contribution of CH═CH on decomposition enthalpies and decomposition entropies can be 5016 J/mol and 17.42 J/mol/K.

Δ_{Dec}Hₘ=5016C_{CH═CH}+81092 Eq. 61

Δ_{Dec}Sₘ=17.42_{CH═CH}+207.317 Eq. 62

Where C_{CH═CH} can be the number of CH═CH functional group in FAMEs.

The contribution of the functional groups in FAMEs on the decomposition enthalpies and entropies of UICs formed by urea and FAMEs can be shown in Table 5.

TABLE 5
Effect of the Functional Group on the Decomposition Enthalpies
and Decomposition Entropies of UICs Formed by Urea and FAMEs

| | CH₃— | —CH₂— | —CH═CH— | —COO— |
|---|---|---|---|---|
| Decomposition | 6374.5 | 6374.5 | 5016 | −21596.7 |
| Enthalpy (J/mol) | | | | |
| Decomposition | 11.684 | 11.684 | 17.42 | 19.278 |
| Entropy (J/mol/K) | | | | |

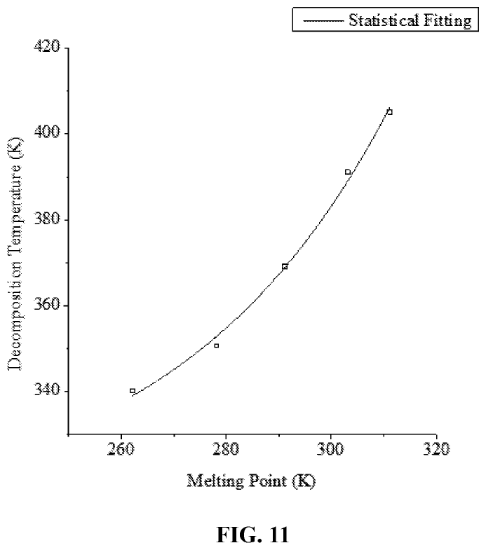

The difference between the decomposition temperatures of UICs formed by urea and saturated fatty acid and the melting point of fatty acid can be approximately constant. According to the existing data on decomposition temperatures of UICs formed by urea and saturated FAMEs and the melting point of saturated FAMEs, the relationship between the decomposition temperatures and melting points can be established (as shown in Eq. 63 and FIG. 11).

T_{D}=310.51016+0.04364 exp(0.02473Tₘ) Eq. 63

Where T_{D} and Tₘ, can be the decomposition temperature and melting point, respectively.

According to the group contribution values of the functional groups, the decomposition temperatures and decomposition enthalpies can be calculated (as shown in Table 6). The predicted decomposition temperatures and decomposition enthalpies can be only used for UICs with the unknown decomposition temperatures and the decomposition enthalpies.

TABLE 6
Decomposition Temperatures and Decomposition Enthalpies of UICs Formed by Urea and FAMEs

| | C12:0 | C14:0 | C16:0 | C16:1 | C18:0 | C18:1 | C18:2 | C18:3 |
|---|---|---|---|---|---|---|---|---|
| Predicted | 344.2 | 369.93 | 389.83 | 362.8 | 405.68 | 381.9 | 356.81 | 330.31 |
| Decomposition | | | | | | | | |
| Temperature | | | | | | | | |
| (K) | | | | | | | | |
| Detected | 350.65 | 369.15 | 391.15 | | 405.15 | 383.15 | | |
| Decomposition | | | | | | | | |
| Temperature | | | | | | | | |
| (K) | | | | | | | | |
| Predicted | 54897 | 67646 | 80395 | 72662 | 93144 | 85411 | 77678 | 69945 |
| Decomposition | | | | | | | | |
| Enthalpy | | | | | | | | |
| (J/mol) | | | | | | | | |
| Detected | | 67298 | 81092 | | 92796 | 86108 | | |
| Decomposition | | | | | | | | |
| Enthalpy | | | | | | | | |
| (J/mol) | | | | | | | | |

In urea inclusion fractionation, the materials can be usually solvent (methanol, ethanol, or propanol), urea and FAMEs. According to the modified UNIFAC model, the functional groups can be methanol, CH₃/CH₂/CH, OH(p)/OH(s), CH₂(C═O)O, CH═CH and Urea. The group volume parameters and group surface area parameters can be listed in Table 7.

TABLE 7
Parameters of Group Volume and Group Surface Area

| | Group Volume | Group Surface Area |
|---|---|---|
| Group Type | Parameter (δₖ) | Parameter(Qₖ) |
| CH₃ | 0.6325 | 1.0608 |
| CH₂ | 0.6325 | 0.7081 |
| CH | 0.6325 | 0.3554 |
| CH₃OH | 0.8585 | 0.9938 |
| OH(p) | 1.2302 | 0.8927 |
| OH(s) | 1.063 | 0.8663 |
| CH═CH | 1.2832 | 1.2489 |
| CH₂(C═O)O | 1.27 | 1.4228 |
| Urea | 3.1278 | 1.8684 |

Through some of the group interaction parameters can be reported (as shown in Table 8), the group interaction parameters of urea with methanol, urea with CH₂(C═O)O, urea with OH(p)/OH(s), urea with CH₃/CH₂/CH and urea with CH═CH can be still unknown. The unknown group interactions parameters can be determined in this disclosure.

TABLE 8
Group Interaction Parameters

| | | Urea | CH3OH | CH3/CH2/CH | OH(p)/OH(s) | CH═CH | (C═O)°CH3 |
|---|---|---|---|---|---|---|---|
| Urea | A | 0 | −7.5248 | 301.3 | −285.3 | 1159.3 | −211 |
| | B | 0 | 0.8026 | 0.1894 | 0.5528 | −4.812 | 0.103 |
| | C | 0 | 0 | 0 | 0 | 0 | 0 |
| CH3OH | A | 30.881 | 0 | 82.593 | 346.31 | −96.297 | 299.23 |
| | B | −0.00171 | 0 | −0.4857 | −2.4583 | 0.6304 | −1.2703 |
| | C | 0 | 0 | 0 | 0.00293 | −0.0018 | 0 |
| CH3/CH2/CH | A | 308.3 | 2409.4 | 0 | 2777 | 189.66 | 98.656 |
| | B | 0.6466 | −3.0099 | 0 | −4.677 | −0.2723 | 1.9294 |
| | C | 0 | 0 | 0 | 0.00155 | 0 | −0.0031 |
| OH(p)/OH(s) | A | 961.8 | −1218.2 | 1606 | 0 | 1566 | 973.8 |
| | B | −2.2579 | 0.0874 | −4.746 | 0 | −5.809 | −5.633 |
| | C | 0 | 0.0106 | 0.00092 | 0 | 0.0052 | 0.00769 |
| CH═CH | A | −13145.9 | −628.07 | −95.418 | 2649 | 0 | 980.74 |
| | B | 46.0959 | 10 | 0.06171 | −6.508 | 0 | −2.4224 |
| | C | 0 | −0.015 | 0 | 0.00482 | 0 | 0 |
| (C═O)°CH3 | A | −473 | 294.76 | 632.22 | 310.4 | −582.82 | 0 |
| | B | 0.1916 | 0.3745 | −3.3912 | 1.538 | 1.6732 | 0 |
| | C | 0 | 0 | 0.00393 | −0.0049 | 0 | 0 |

According to the model, the solubility of some urea inclusion compounds formed by urea and FAMEs can be predicted and measured. The predicted results can be close to the measured data, which shows this model can be good.

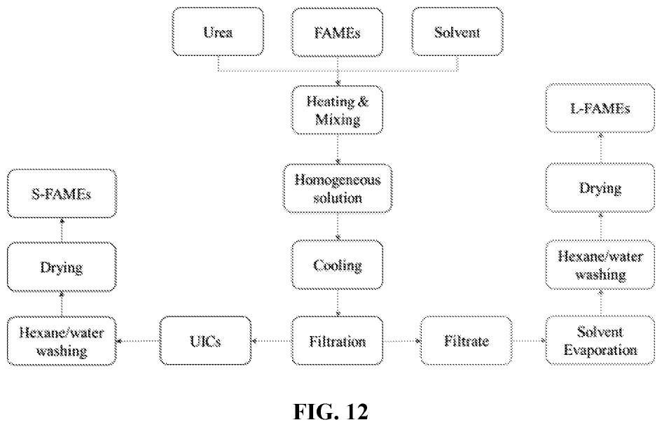

The scheme of the urea inclusion process can be shown in FIG. 12. The solvents used in this disclosure can include methanol, ethanol, and/or n-propanol. The weight of FAMEs can be fixed at 50 g in all experiments. However, the weights of urea and solvents can vary according to their mass ratios concerning FAMEs. FAMEs from vegetable oils can be produced as shown in the previous section, but FAMEs from chicken fat and waste cooking oil can be purchased from a local biodiesel company. The mass ratio of urea to FAMEs varied from 0 to 1 while the mass ratio of solvent to FAMEs varied from 3 to 5. The mixture of urea, FAMEs, and solvent in a 1-L flask can be heated to form a homogenous solution with the maximum temperatures limited to 5° C. less than the boiling points of the solvents to prevent evaporation. Then, the solution can be cooled down to room temperature 0° C. using ice, and the resulting solid/liquid mixture can be separated by filtration. 200 mL hexane can be used to rinse the UICs to minimize the L-FAMEs yield loss. The solvent in the liquid filtrate can be removed by a rotary evaporator. The residual can be placed into a separatory funnel, and 200 mL of distilled water can be added with mixing to remove residual urea and solvent, followed by removal of the water layer after 0.5-hour settling. This step can be repeated several times until the water layer became clear. The bottom layer can be drained, and the top layer can be put into a rotary evaporator to remove the moisture to obtain L-FAMEs. The solid residual in the filtration can be mixed with hot water to decompose the UICs. Then, the mixture can be transferred to a separation funnel, and 150 ml hexane can be added to maximize the recovery of the FAMEs. The hexane can be recovered in a rotary evaporator after the hexane layer can be transferred, and the residual liquid can be the S-FAMEs. The compositions of L-FAMEs and S-FAMEs can be analyzed according to the steps in the following section.

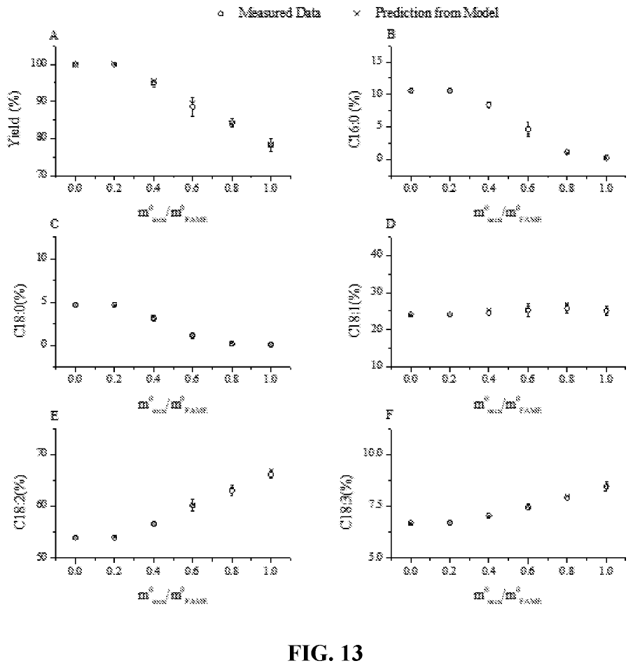

As shown in FIGS. 13A-F, the thermodynamic model can accurately predict the yield of FAMEs and the composition of FAMEs. The urea to FAME mass ratios (R_{U/F}) varied between 0 and 1 in 0.2, with a fixed mass ratio of urea to methanol (R_{U/M}) at 4. R_{U/F} had little effect on the urea inclusion fractionation when less than 0.2. The composition and yield of FAME in the liquid phase did not change when R_{U/F} can be in this range. For R_{U/F} above 0.4, the yield of FAME in the liquid phase decreased (L-FAMEs), and the composition of L-FAMEs changed with R_{U/F}. Meanwhile, the saturated FAME in L-FAMEs decreased with the increased R_{U/F}, but the polyunsaturated FAME. For the monounsaturated FAME, methyl oleate, the fraction of methyl oleate in the liquid phase had a maximum value when R_{U/F} can be 0.8.

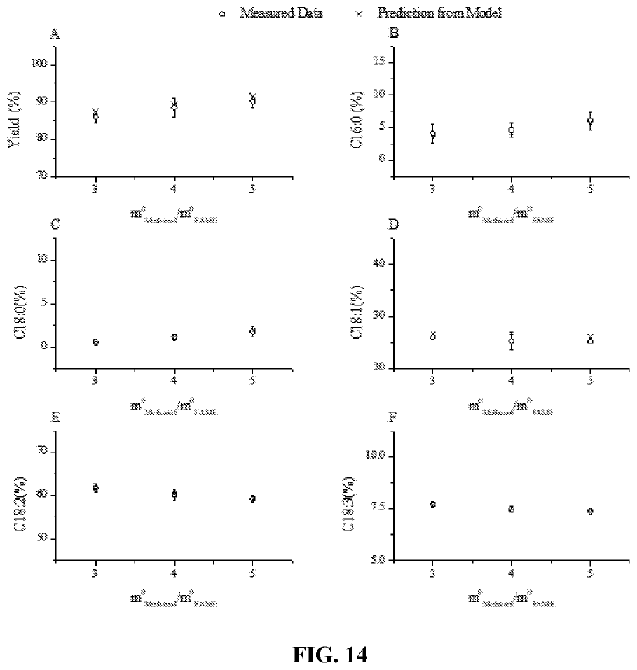

As shown in FIGS. 14A-F, the thermodynamic model also can accurately predict the yield and composition changes in urea inclusion with various R_{U/M}. R_{U/M} varied between 3 and 5 in increments of 1 with a fixed R_{U/M} of 0.6. With the increase in R_{U/M}, the yield of FAME in L-FAMEs increased. The saturated FAME in L-FAMEs increased, and the unsaturated FAME in L-FAMEs decreased with the increase in the mass ratios of methanol to FAME.

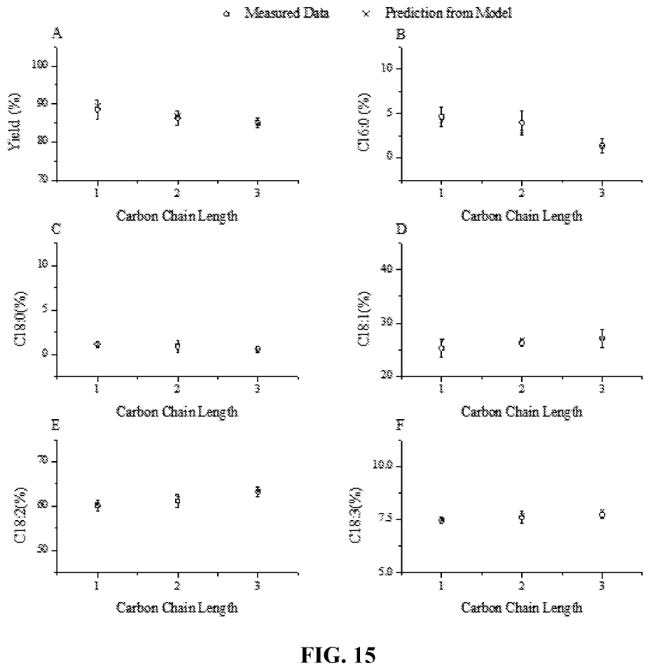

Methanol, ethanol, and n-propanol can be used to examine the effect of the carbon chain length of alcohols on urea inclusion fractionation, and the mass ratio of solvent to urea to FAMEs to can be fixed at 4:0.6:1. The comparison of the prediction and the experimental data can be shown in FIGS. 15A-F. The fractions of the saturated FAME in LFAMEs decreased with the increase in the carbon chain length of alcohols. However, the fractions of the unsaturated FAME in L-FAMEs increased with the increase in the carbon length of alcohols.

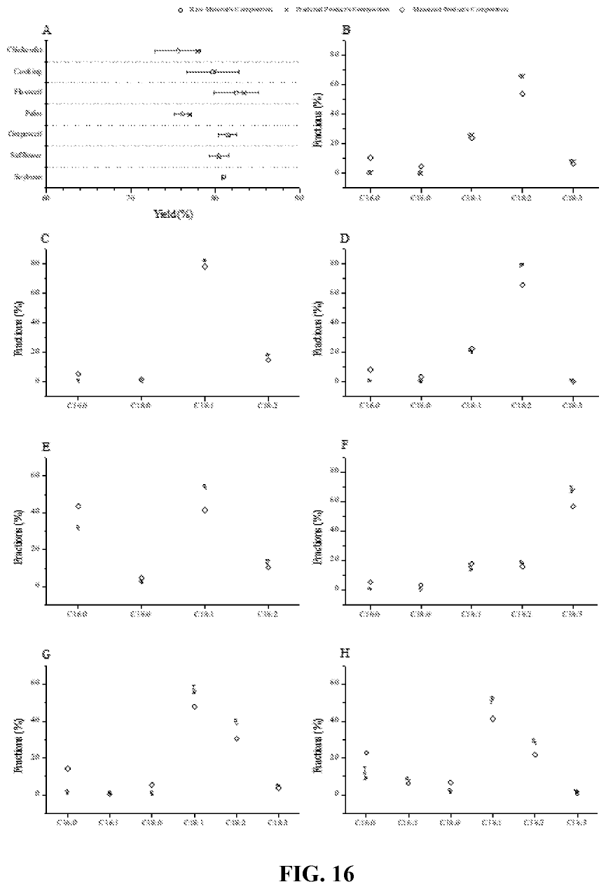

The mass ratio of urea to FAME to methanol can be fixed at 1:1:4, and FAMEs produced from soybean oil, safflower oil, grape seed oil, flaxseed oil, cooking oil, chicken fat, and palm oil can be examined. The comparison of the prediction by the thermodynamic model and the experimental data can be shown in FIGS. 16A-H.

The urea inclusion fractionation reduced the concentration of the saturated FAMEs in all the tested mixtures of FAME. The yields of L-FAMEs ranged from 75 to 85%. At the same mass ratio of urea to FAME to solvent, the yields of L-FAMEs can be low when the saturated FAME can be high in the raw materials.

The thermodynamic model can be proposed to predict the urea inclusion fractionation based on the phase equilibrium, and the modified UNIFAC model predicted the non-ideal behavior. The model can accurately predict the yield and composition changes during the urea inclusion fractionation compared to the experimental data. In one or more embodiments, the predicted yield and composition changes can be used to select a FAME source for producing the winter-season diesel fuels. The FAME source can include composition and/or other physical and chemical information. The urea inclusion fractionation can include operation data that can include any relevant operating information. In one or more embodiments, the model can accurately predict the yield and composition changes during the urea inclusion fractionation using FAME source information and/or operation data. In one or more embodiments, the model predictions can be used to select one or more FAME sources to be used for producing winter-season biodiesel.

All patents and patent applications, test procedures (such as ASTM methods, UL methods, and the like), and other documents cited herein can be fully incorporated by reference to the extent such disclosure can be not inconsistent with this disclosure and for all jurisdictions in which such incorporation can be permitted.

Certain embodiments and features can be described using a set of numerical upper limits and a set of numerical lower limits. It should be appreciated that ranges including the combination of any two values, e.g., the combination of any lower value with any upper value, the combination of any two lower values, and/or the combination of any two upper values can be contemplated unless otherwise indicated. Certain lower limits, upper limits and ranges appear in one or more claims below.

The foregoing has also outlined features of several embodiments so that those skilled in the art can better understand the present disclosure. Those skilled in the art should appreciate that they can readily use the present disclosure as a basis for designing or modifying other methods or devices for carrying out the same purposes and/or achieving the same advantages of the embodiments disclosed herein. Those skilled in the art should also realize that such equivalent constructions do not depart from the spirit and scope of the present disclosure, and that they can make various changes, substitutions, and alterations herein without departing from the spirit and scope of the present disclosure, and the scope thereof can be determined by the claims that follow.

We claim:

1. A method for predicting yield and composition changes during urea inclusion fractionation to select a fatty acid methyl ester source for biofuel, the method comprising:
providing a solid urea inclusion compound having a first temperature;
heating the solid urea inclusion compound to a second temperature sufficient to decompose the solid urea inclusion compound;
recording a change in enthalpy and a change in entropy to provide a first recorded change in enthalpy and a first recorded change in entropy from the first temperature to the second temperature;
decomposing the solid urea inclusion compound at the second temperature to produce a mixture of solid urea and liquid fatty acid methyl ester;
recording a change in enthalpy and a change in entropy while decomposing the solid urea inclusion compound at the second temperature to provide a second recorded change in enthalpy and a second recorded change in entropy;
heating the mixture of the solid urea and liquid fatty acid methyl ester to a third temperature sufficient to melt the urea, wherein the third temperature is greater than the second temperature;
recording a change in enthalpy and a change in entropy to provide a third recorded change in enthalpy and a third recorded change in entropy from the second temperature to the third temperature;
melting the solid urea at the third temperature to produce a mixture of liquid urea and liquid fatty acid methyl ester;
recording a change in enthalpy and a change in entropy while melting the solid urea to provide a fourth recorded change in enthalpy and a fourth recorded change in entropy;
cooling the mixture of the liquid urea and liquid fatty acid methyl ester to the first temperature;
recording a change in enthalpy and a change in entropy to provide a fifth recorded change in enthalpy and a fifth recorded change in entropy from the third temperature to the second temperature;
cooling the mixture of liquid urea and liquid fatty acid methyl ester to a fourth temperature;
recording a change in enthalpy and a change in entropy to provide a sixth recorded change in enthalpy and a sixth recorded change in entropy from the second temperature to the fourth temperature;
calculating a thermodynamic model using the six recorded changes in enthalpies and six changes in entropies;
using the thermodynamic model to predict a yield and composition of the solid urea inclusion compound; and
selecting a fatty acid methyl ester source using the predicted yield and composition.

2. The method of claim 1, wherein calculating the thermodynamic model using the six recorded changes in enthalpy and six changes in entropy comprises:
ΔH_{m,i}^{o}(T,P)=∫_{T}^{T}^{_{d,i}}ΔC_{p,m,i}dT+∫_{T}_{_{d,i}}^{T}^{_{m,u}}(βΔC_{p,m,u})dT+βΔ_{fus}H_{m,u}+Δ_{dec}H_{m,i}
ΔS_{m,i}^{o}(T,P)=∫_{T}^{T}^{_{d,i}}(ΔC_{p,m,i}/T)dT+∫_{T}_{_{d,i}}^{T}^{_{m,u}}(βΔC_{p,m,u}/T)dT+βΔ_{fus}H_{m,u}/T_{m,u}+Δ_{dec}H_{m,i}/T_{d,i}

3. The method of claim 1, wherein using the thermodynamic model to predict a yield and composition of the solid urea inclusion compound comprises:
R ln(γᵢ^{L}xᵢ^{L}(γ_{U}^{L}x_{U}^{L})^{β})=∫_{T}^{T}^{_{d,i}}(ΔC_{p,m,i}/T)dT−(∫_{T}^{T}^{_{d,i}}ΔC_{p,m,i}dT)/T+∫_{T}_{_{d,i}}^{T}^{_{m,u}}(βΔC_{p,m,u}/T)dT−(∫_{T}_{_{d,i}}^{T}^{_{m,u}}(βΔC_{p,m,u})dT)/T+βΔ_{fus}H_{m,u}(1/T_{m,u}−1/T)+Δ_{dec}H_{m,i}(1/T_{d,i}−1/T)

4. The method of claim 1, wherein the predicted yield and composition comprise the yield and composition of fatty acid methyl esters in a liquid phase.

5. The method of claim 1, wherein selecting a fatty acid methyl ester source using the predicted yield and composition comprises using fatty acid methyl ester source composition and operation data to predict the yield and composition of fatty acid methyl esters in a liquid phase.

6. The method of claim 1, wherein the second temperature is the decomposition temperature of the solid urea inclusion compound.

7. The method of claim 1, wherein the third temperature is the melting point of the solid urea.

8. The method of claim 1, wherein the fourth temperature is an arbitrary temperature that is less than the second temperature.
